(19) United States
(12) Patent Application Publication
(10) Pub. No.: US 2010/0089408 A1
(43) Pub. Date: Apr. 15, 2010

(54) ANTIMICROBIAL BLOOD PRESSURE CUFF

(76) Inventors: Elizabeth McCaughey, New York, NY (US); Thomas Higgins, Lattingtown, NY (US); Theodore Shlisky, Kings Park, NY (US)

Correspondence Address: GOODWIN PROCTER LLP;ATTN: PATENT ADMINISTRATOR, 620 Eighth Avenue, NEW YORK, NY, 10018, US

(21) Appl. No.: 12/449,464

(22) PCT Filed: Feb. 7, 2008
(86) PCT No.: PCT/US08/01606

§ 371 Date: Nov. 6, 2009

Related U.S. Application Data
(60) Provisional application No. 60/899,937, filed on Feb. 7, 2007.

(51) Int. Cl. A61B 19/02; A61F 15/00

(52) U.S. Cl. 128/846; 206/440

(57) ABSTRACT

This invention is directed to blood pressure cuff liners for reducing the possibility of infection to patients as a result of blood pressure measurements, and to methods of reducing such infections. The liners of the present invention provide a barrier between the patient and the blood pressure cuff used during blood pressure measurement, and also comprise chemical compounds that are able to eradicate most bacterial and viral pathogens, as well as fungi and molds. The liners of this invention are made to be disposable, but can be reused without risk to patients, because its pathogen-eradicating qualities protect patients from infection.

### CROSS REFERENCE TO RELATED APPLICATIONS

This application claims the benefit of priority of U.S. provisional application 60/899,937 filed on Feb. 7, 2007 which is incorporated by reference herein.

### BACKGROUND OF THE INVENTION

1. Field of the Invention

This invention is directed to blood pressure cuff liners for reducing the possibility of infection to patients as a result of having blood pressure measurements taken, and to methods of reducing such infections. The liners of the present invention comprise: chemical compounds that are able to eradicate most bacterial and viral pathogens, as well as fungal and molds. The liners of this invention are made to be disposable, but can be reused without risk to patients, because its pathogen-eradicating qualities protect patients from infection.

2. Background Information

It is estimated that one out of every twenty patients that enters a hospital picks up an infection during the hospital stay. As noted in “Dirty Hospitals” (AARP Bulletin, Vol. 48, No. 1, p. 12), about two million patients pick up hospital infections every year, and more than 90,000 of these patients die. Nationwide, hospital infections are the eighth-leading cause of death. Additionally, hospital infections lead to extended hospital stays for the patients, and increased costs to both patient and the hospital.

A common source of hospital infections is inadequate hygiene that can be a result of dirty hands, improperly cleaned equipment, unclean rooms, and lax procedures. For example, according to the article “Doctors, Wash Your Hands,” (Los Angeles Times, Feb. 3, 2007) commonly used devices such as stethoscopes, blood pressure cuffs, and pulse oximeters can spread bacteria if not properly disinfected. A recent study in the journal Infection Control and Hospital Epidemiology (September 2006) indicated that 77% of blood pressure cuffs in a French teaching hospital were contaminated with bacteria on the interior surface of the blood pressure cuff, the surface that comes into contact with a patient's arm when blood pressure measurements are taken. The researchers who conducted the study proposed that hospital staff clean the blood pressure cuffs with detergent several times each shift. However, such a procedure is cumbersome and easy to disregard during busy times. Also, typical detergents or reagents have limited effectiveness and/or duration of effectiveness in killing microorganisms.

One solution to mitigate infections due to contaminated blood pressure cuffs is to use a liner. However, existing liners, even liners that come pre-packaged, are easily contaminated during handling by caregivers, because they only function as barriers to and/or absorbers of contamination. For example U.S. Pat. No. 6,364,843, U.S. Pat. No. 6,525,238, U.S. Pat. No. 4,967,758, U.S. Pat. No. 5,251,646, U.S. Pat. No. 4,548,249, U.S. Pat. No. 5,228,448, U.S. Pat. No. 5,669,390, and U.S. Pat. No. 5,513,643 all disclose versions of disposable liners that either act as a barrier between the patent and the blood pressure cuff, or act to absorb and contain fluids and contamination from one patient so that the blood pressure cuff itself does not become contaminated. However, liners that work in this fashion can become contaminated if touched by contaminated hands of caregivers, and the liner can subsequently pass the contamination along to the patient. Indeed, research shows that physicians, for example, fail to clean hands 52% of the time before touching patients. In some institutions, the frequency is even higher. Furthermore, even if a caregiver does wash hands (or use gloves), the originally clean hands can be easily re-contaminated when the caregiver touches objects or surfaces, such as when handling privacy curtains in a treatment room. The caregiver can then pass the contamination to a patient when handling a blood pressure cuff liner. The presently available liners, therefore, do not adequately protect the patient from contamination that originates from sources other than the blood pressure cuff itself. Furthermore, while U.S. Pat. No. 5,513,643 discloses liners containing general sanitizers, general sanitizers are not always suitable for application to liners, and are not specifically effective against some of the most pertinent and pernicious sources of infections in hospitals, such as methicillin-resistant Staphylococcus aureus. Additionally, general sanitizers are not necessarily designed to have the prolonged duration of effectiveness that would be needed to manufacture a blood pressure cuff liner that can be used months or years after manufacture.

In view of the above-described problems associated with infections attributable to hospital stays, and particularly, the frequent use of common medical devices, such as blood pressure cuffs, which can be the source of contamination and infection, it would be desirable to minimize the likelihood that such common devices carry and spread infections.

Accordingly, it is a first object of this invention to provide an easy-to-use, disposable blood pressure cuff liner to minimize patient infections from blood pressure cuffs.

It is one feature of the blood pressure cuff liner of the present invention that it can be made from various materials, such as tissue paper, polypropylene and/or other materials, either as single layers or in multiple layers (i.e., as laminates). Such materials act as a barrier between the patient and the blood pressure cuff, and can be made to be disposable, or durable enough for multiple uses.

It is another feature of the blood pressure cuff liner of this invention that the liner comprise a fastening system. The fastening system allows for easy and convenient use of the liners, making it more likely that caregivers will use the liners when making blood pressure measurements.

It is another feature of the blood pressure cuff liners of the present invention that the blood pressure cuff liners comprise an antimicrobial compound with disinfecting ability to kill or reduce the occurrence of microorganisms that come into contact with the liner. The anti-microbial compounds which may be employed in this invention include, but are not limited to organosilane compounds, such as 3-(trihydroxysilyl) propyl-dimethyloctadecyl ammonium chloride, stabilized in water. Such compounds are effective against a broad spectrum of stubborn pathogens, such as methicillin-resistant Staphylococcus aureus. Additionally, such compounds have a residual disinfecting ability that allow a liner to be effective to protect the patient if the liner is used more than once, if the liner is not immediately used after it has been removed from its packaging, or if the liner has been handled by contaminated hands of the caregiver. A further advantage of the organosilane compounds of this invention is that they adhere well to most surfaces, including a wide array of liner materials.

It is another feature that the antimicrobial compound of the present invention can be combined with a wetting agent. Non-ionic wetting agents are preferred. A preferred combination of anti-microbial agent and wetting agent is the combination of 3-(trihydroxysilyl) propyl-dimethyloctadecyl ammonium chloride with a 10% additive of a non-ionic wetting agent consisting of ethoxylated alcohol and glycol ethers category N230.

It is a second object of this invention to provide an easy-to-use, disposable blood pressure cuff liner as a kit or package to allow convenient dispensing of the liners.

It is a third object of this invention to provide a method of reducing the possibility of infection from use of blood pressure cuffs.

Other objects, features and advantages of the blood pressure cuff liners, kits, and methods of this invention will be apparent to those skilled in the art in view of the detailed description of the invention set forth herein.

### SUMMARY OF THE INVENTION

A blood pressure cuff liner to minimize patient infections from blood pressure cuffs comprising:

an organosilane antimicrobial compound or a silver containing compound or particle; and,

a substrate.

A blood pressure cuff liner comprising:
3-(trihydroxysilyl) propyl-dimethyloctadecyl ammonium chloride or a silver nanoparticle;
a fastening system; and,
a substrate.

A kit comprising:

a plurality of blood pressure cuff liners comprising an organosilane antimicrobial compound or a silver containing compound or particle, a fastening system, and a substrate, wherein the blood pressure cuff liners are removably connected to one another to allow dispensing of the blood pressure cuff liners.

A method of reducing the possibility of infection from use of blood pressure cuffs comprising providing a blood pressure cuff liner comprising an organosilane compound or a silver containing compound or particle.

### BRIEF DESCRIPTION OF THE DRAWINGS

FIG. 1 depicts a top view of one embodiment of this invention.

FIG. 2 depicts the embodiment of FIG. 1 in preparation for use on a patient.

FIG. 3 depicts a bottom view of one embodiment of this invention.

FIG. 4 depicts the embodiment of FIG. 3 as it is being affixed to the inner surface of a blood pressure cuff.

FIG. 5 depicts an embodiment of this invention comprising a plurality of liners.

FIG. 6 depicts an embodiment of this invention comprising a plurality of liners and a package.

### DETAILED DESCRIPTION OF THE INVENTION

This invention will be further understood in view of the following detailed description. The present invention provides an easy-to-use and disposable liner for use with blood pressure cuffs to minimize the likelihood of infections caused by contaminated blood pressure cuffs, or from the contaminated hands of caregivers who handle blood pressure cuff liners. The present invention takes account of the impracticability of frequently and thoroughly cleaning such frequently used devices with detergents or other reagents, which can be cumbersome and where such procedures can be limited in effectiveness. The solution provided by the present invention makes use of relatively inexpensive and disposable materials in combination with antimicrobial compounds to provide an easy-to-use liner that acts as a barrier between a patient and a blood pressure cuff, and also acts to kill or reduce residual contamination on the blood pressure cuff or contamination from the hands of caregivers. The present invention is described in greater detail herein relative to non-limiting embodiments of the invention, and with reference to the drawings.

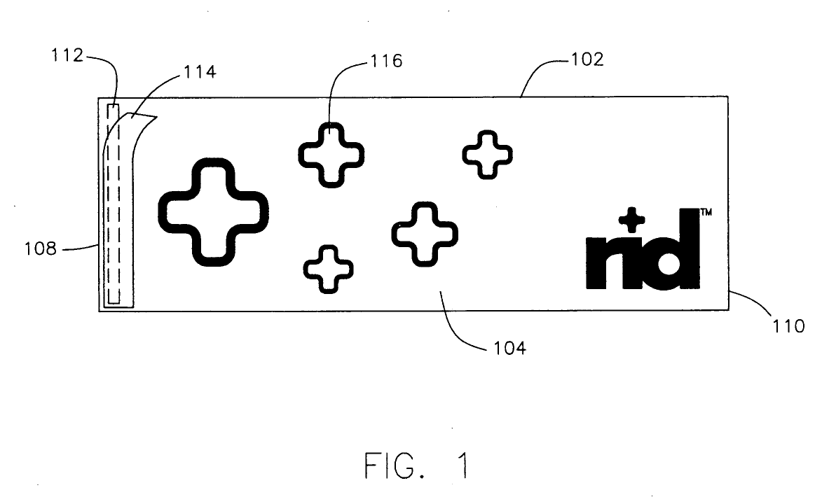

Referring now to FIG. 1, there is depicted a view of an embodiment of the blood pressure cuff liner (102) of the present invention, having a top-side surface (104) and an opposing bottom-side surface (not shown in FIG. 1), and a first short end (108) and second short end (110). The liner (102) has an adhesive tape strip (112) located at the first short end (108) of the liner, so that the liner (102) can be easily attached to a patient during a blood pressure measurement. The adhesive tape strip (112) further comprises a removable cover sheet (114) that can be peeled off just prior to use. The liner (102) may also have embossing, designs, patterns, or other decorative markings or markings (116) to describe source of origin or other features of the liner. Such markings can appear on the top-side surface and/or the bottom-side surface of the liner.

The exact dimensions of the liner can vary, depending on the dimensions of the blood pressure cuff that will be used and characteristics of the patient. The dimensions of the liner should be at least as wide, or slightly wider, than the width of the blood pressure cuff. The length of the liner should be sufficient to wrap completely around the patient's arm, and serve as a barrier between the patient's arm and the blood pressure cuff. A typical width of the liner is about 5.5 inches. Lengths will vary to meet the needs of children, adults, and the obese, preferably ranging from about 12 inches to about 36 inches.

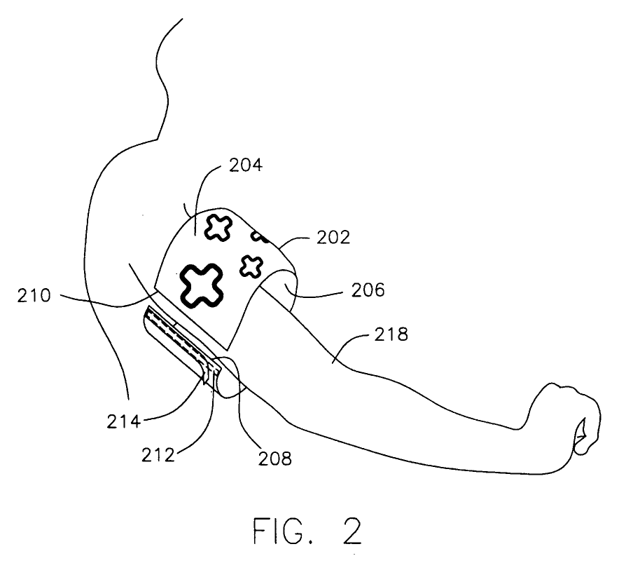

FIG. 2 shows a liner of FIG. 1 as it is used on a patient in preparation for a blood pressure measurement. The liner (202) has an adhesive tape strip (212) and cover sheet (214) for the adhesive tape strip (212). The adhesive tape strip is affixed to the top-side surface (204) of the liner (202) at the liner's first short end (208). To attach the liner (202) to the patient's arm (218), the removable cover sheet (214) is peeled away to expose the adhesive tape strip (212) underneath the cover sheet. The adhesive tape strip (212) can be secured to the second short end (210) of the bottom-side surface (206) of the liner (202). Once the liner (202) is attached around a patient's arm, the caregiver then attaches the blood pressure cuff over the top of the liner (202) to measure the patient's blood pressure.

While FIGS. 1 and 2 disclose an adhesive tape strip (112 and 212, respectively) for securing the liner (102, 202) to a patient's arm, any fastening system can be used. For example, a liner within the scope of the present invention can be made without an adhesive strip, requiring the caregiver to use medical tape, clips, or other ways to secure the liner to the patient's arm. Alternatively, the liner can comprise a loop and hook system (i.e., Velcro® brand fasteners), buttons, snaps, hooks, or other fastening systems or devices to secure the liner around a patient's arm. The advantage of the adhesive strip is that it is convenient to use, comfortable for the patient, and relatively inexpensive.

FIG. 1 discloses the adhesive tape strip (112) located at the first short end (108) of the liner (102), extending across a major portion of the first short end (108). However, the exact location and length of the adhesive tape strip (112) is not important, as long as it serves to secure the liner (102) for use. Additionally, the number of adhesive tape strips (112), or other fastening systems or devices used to secure the liner (102) to a patient's arm is not critical, as long as the liner (102) can be used effectively. For example, instead of a single long adhesive tape strip (112) located at a first short end (108) of the liner (102), the liner (102) can be made with one or more smaller adhesive tape strips (112) affixed to either the top-side surface (104), the bottom-side surface (106), or both.

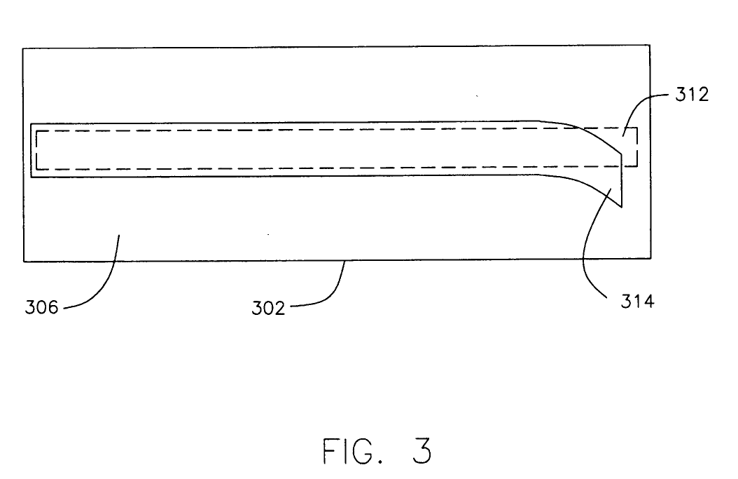

FIG. 3 shows the bottom-side surface (306) of a liner (302) of an additional embodiment of this invention. In this embodiment, the adhesive tape strip (312) is affixed to the bottom-side surface (306) of the liner (302), to enable attachment of the liner (302) to a blood pressure cuff rather than directly to a patient's arm. The adhesive tape strip (312) has a removable cover sheet (314) that can be peeled away prior to attachment to a blood pressure cuff. As shown in FIG. 3, the adhesive tape strip (312) can be affixed to the liner (302) lengthwise, along a central portion of the liner (302). However, the placement and dimensions of the adhesive tape strip (312) can vary, as can the number of adhesive tape strips (312). Alternatively, the liner (302) can be made without an adhesive tape strip (312), in which case a caregiver using the liner (302) can attach the liner (302) to a blood pressure cuff using medical tape, clips, or some other fastening system or device. Furthermore, instead of an adhesive tape strip (312), the liner (302) in FIG. 3 can comprise a loop and hook system (i.e., Velcro® Brand fasteners), buttons, snaps, hooks, or other fastening system or devices to secure the liner (302) to a blood pressure cuff.

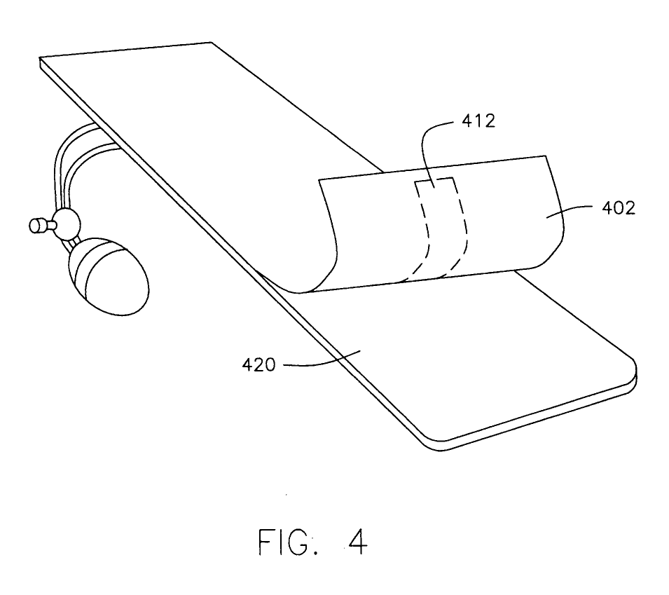

FIG. 4 shows how a liner of FIG. 3 can be attached to a blood pressure cuff (420). As shown with respect to the liner (402) in FIG. 4, the cover sheet (not shown in FIG. 4) of the adhesive tape strip (412) has already been removed, so that the adhesive tape strip (412) can adhere to the inner surface (422) of the blood pressure cuff (420).

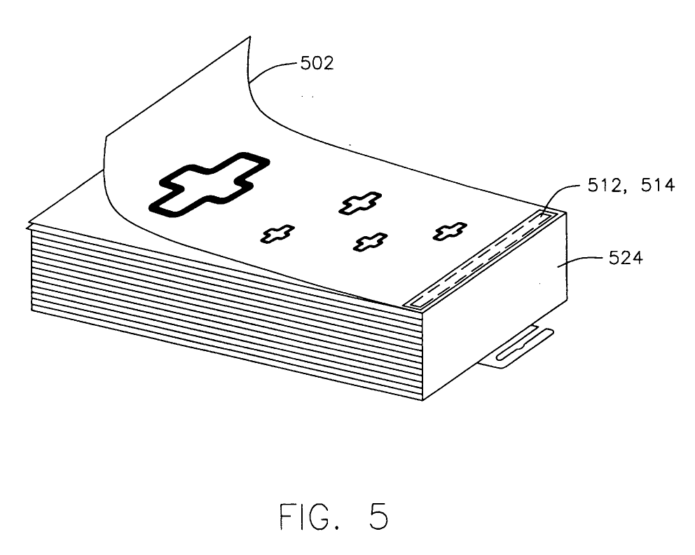

FIG. 5 shows an embodiment of the present invention comprising a plurality of liners (502) assembled for easy and convenient dispensing. The plurality of liners (502) are held together by a spine (524). Also shown in FIG. 5 is the adhesive tape strip with cover sheet (512, 514).

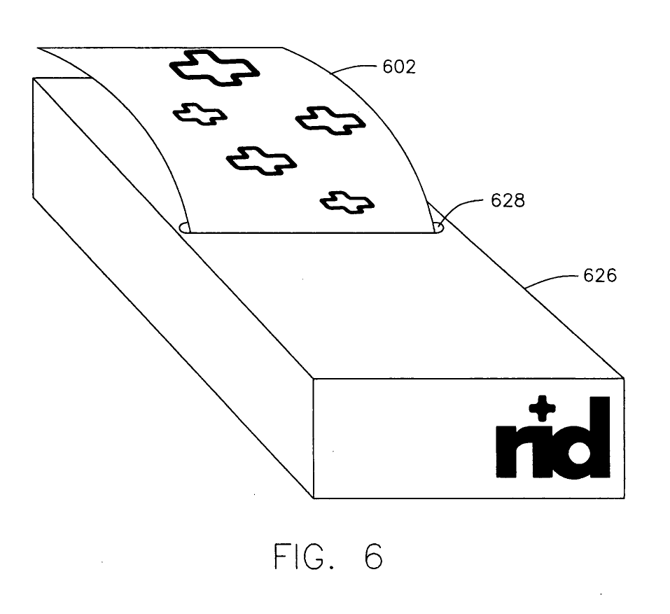

FIG. 6 shows an additional embodiment comprising a plurality of liners (602) together with a package (626) having an opening (628) for easy and convenient dispensing. Other assemblies of liners and liners with packaging or other dispensing devices are within the scope of the present invention. For example, liners can be packaged and dispensed on a roll, with perforations between the liners. Additionally, liners can be packaged and dispensed on a hard-backed pad with a loop at the top that can be hung from the blood pressure trolley, and with perforations at one end of the liners to allow the liners to be torn off the pad for convenient use. Furthermore, liners can be packaged and dispensed as a plurality of flat folded and stacked liners, in a box, with an opening at the top of the box, similar to a box of tissue paper. Other packaging and/or dispensing configurations for the blood pressure cuff liners of this invention are possible.

In each of the above-mentioned embodiments, the liner (102, 202, 302, 402, 502, and 602) contains a substance (i.e., an active agent, active ingredient, anti-microbial compound, or anti-microbial) that can kill or reduce the number of microorganisms that come into contact with the liner. The anti-microbial compounds which may be employed in this invention include, but are not limited to organosilane compounds as described in U.S. Pat. No. 5,959,014, U.S. Pat. No. 6,221,944, and U.S. Pat. No. 6,632,805, each of which is incorporated herein by reference. Additional anti-microbials which may be employed in this invention include silver containing organic or inorganic compounds or particles, including, but not limited to ionic forms of silver and silver nanoparticles.

Organosilane compounds can be applied to the liner material or substrate as either an aqueous or non-aqueous form. A preferred organosilane compound is the 3-(trihydroxysilyl) propyl-dimethyloctadecyl ammonium chloride that is available from NBS Technology, LLC under the trade name GOLDSHIELD, as 5% 3-(trihydroxysilyl) propyl-dimethyloctadecyl ammonium chloride, stabilized in water. This compound consists of a fat or lipid soluble portion and a water-soluble portion containing a positive charge. Without wishing to be bound by any one theory, it is believed that the positive charge attracts negatively charged microorganisms. When these microorganisms come in contact with the pathogen-killing ingredient, it disrupts the organism membrane on a nano-molecular scale, and physically “stabs” it, electrically pulls ions out, and denatures proteins. This active ingredient is effective in eradicating many gram negative and gram positive bacteria, including methicillin-resistant Staphylococcus aureus (MRSA), Clostridium difficile, Escherichia coli, Pseudomonas aeruginosa, Acinetobacter calcoaceticus 1, and Streptococcus faecalis, as well as a broad variety of fungi, algae, yeast, and certain viruses, including influenza A, HIV B, and SARS.

When used as the anti-microbial compound for the liners of this invention, 3-(trihydroxysilyl) propyl-dimethyloctadecyl ammonium chloride can be combined with a wetting agent. Non-ionic wetting agents are preferred. A preferred combination of anti-microbial agent and wetting agent is the combination of 3-(trihydroxysilyl) propyl-dimethyloctadecyl ammonium chloride with a 10% additive of a non-ionic wetting agent consisting of ethoxylated alcohol and glycol ethers category N230 (10% denotes the concentration of additive in solution prior to addition to the active agent solution).

The active agent, whether 3-(trihydroxysilyl) propyl-dimethyloctadecyl ammonium chloride or another compound, can be applied to the liners of this invention by spraying, dipping, wiping, exhausting, pressure cylinder application, or other suitable processes. The active agent can be applied to one or both sides of the liner—the side that contacts the blood pressure cuff and/or the side that contacts the patient's arm. The amount of active agent to be applied should result in an effective concentration to be able to kill microorganisms upon contact. For 3-(trihydroxysilyl) propyl-dimethyloctadecyl ammonium chloride, the concentration should range generally from about 0.01% to 5.0% (ratio of active ingredient to weight of total liquid concentrate). A range of about 0.5% to 5.0% is preferred. The exact concentration of active ingredient may vary, depending on the type and nature of active ingredient used and form in which it is applied to the liner substrate, and the type and nature of the liner substrate. Once applied, the active agent solution needs to dry to become effective.

Any type of substrate (i.e. liner material) or combination of substrates suitable to make a liner can be used. For example, liners can be made from three ply tissue paper, non-woven spun bond polypropylene, or a laminate of two ply tissue bonded together with a polymer layer such as polyethylene. Alternate embodiments can be made with tissue paper layered or laminated with other materials. Additional embodiments can be made with other materials suitable for blood pressure cuff liners, either alone, or as layers or laminates. Non-limiting examples of other materials are mentioned in U.S. Pat. No. 4,967,758 and U.S. Pat. No. 5,669,390, which are incorporated herein by reference. Preferred embodiments of the invention will be further described by the following examples which are illustrative only and do not limit the invention.

### Example 1

### Three Ply Tissue Paper Liner

5% 3-(trihydroxysilyl) propyl-dimethyloctadecyl ammonium chloride, stabilized in water, or a combination of 3-(trihydroxysilyl) propyl-dimethyloctadecyl ammonium chloride with a 10% additive of a non-ionic wetting agent consisting of ethoxylated alcohol and glycolethers category N230 is applied by spraying, dipping, wiping, exhausting, or via pressure cylinder release to three ply tissue paper, so that the final amount of active ingredient is from about 0.01% to 5.0% (ratio of active ingredient to weight of total liquid concentrate). The embodiment may include a mechanically embossed or printed design on the paper. The paper liner has an adhesive strip affixed to one short end of the cuff liner, to allow the cuff liner to be secured around the patient's arm. Alternatively, the adhesive strip is affixed down the length of the bottom-side of the liner, to allow the liner to adhere to the inside of the blood pressure cuff. Once the liquid concentrate is applied, it needs to dry to become effective.

### Example 2

### Polypropylene Liner

5% 3-(trihydroxysilyl) propyl-dimethyloctadecyl ammonium chloride, stabilized in water, or a combination of 3-(trihydroxysilyl) propyl-dimethyloctadecyl ammonium chloride with a 10% additive of a non-ionic wetting agent consisting of ethoxylated alcohol and glycolethers category N230 is applied by spraying, dipping, wiping, exhausting, or via pressure cylinder release to a non-woven spun bond polypropylene, so that the final amount of active ingredient is from about 0.01% to 5.0% (ratio of active ingredient to weight of total liquid concentrate). Once the liquid concentrate is applied, it needs to dry to become effective.

The material may also carry an embossed or printed design. This embodiment may also have either an adhesive strip on one end, or an adhesive strip running the length of the bottom-side of the liner.

### Example 3

### Two Ply Tissue Paper Combination Liner

This embodiment has, as one of two distinct layers of the liner, a layer of two ply tissue paper, comprising 10-12 pound basis weight construction on each ply. The second layer of the liner consists of polyethylene having a thickness of about 0.4 mil. to about 0.75 mil. The tissue paper layer and the polyethylene layer are bound together through a heat bonding process. 5% 3-(trihydroxysilyl) propyl-dimethyloctadecyl ammonium chloride with a 10% additive of a non-ionic wetting agent consisting of ethoxylated alcohol and glycolethers category N230 is applied by spraying, dipping, wiping, exhausting, or via pressure cylinder release onto the two ply tissue, at ratios that can vary from 0.01% to 5.0% (ratio of active ingredient to weight of total liquid concentrate). Once the liquid concentrate is applied, it needs to dry to become effective. This embodiment may also be made with embossed or printed designs, and with the adhesive strip affixed to one of the short ends of the liner, or running the length of the bottom-side of the liner.

### Example 4

### Silver Containing Compounds and Particles

This embodiment is made with silver containing compounds or particles as the anti-microbial. Silver is known to be effective against a wide variety of microorganisms, such as bacteria, fungi and yeast. Additionally, silver is active in relatively small quantities. For example, as little as one part per billion of silver may be effective in preventing cell growth of bacteria, and 100 to 3000 parts per million of silver on surfaces can prevent a wide range of pathogens from forming biofilms. The effectiveness of silver as an anti-microbial is described in “The Role of Antimicrobial Silver Nanotechnology” (originally published in Medical Device & Diagnostic Industry Magazine, August 2005; see http://www.devicelink.com/mddi/archive/05/08/005.html), which is incorporated herein by reference.

Preferred embodiments are made with silver nanoparticles. A typical silver nanoparticle can have an average diameter of about 10 nm, but particles sizes and particle size averages can range from under 2 nm to over 20 nm. The anti-microbial effect of silver nanoparticles can persist for over a year. Silver can be applied to surfaces in many ways. Vacuum-sputter coating and plasma-arc deposition technologies can be used to apply vaporized silver to a surface, and ionic plasma deposition can be used to apply ordered layers of silver oxide molecules to surfaces. Additionally, silver nanoparticles can be applied as aqueous or solvent-based solutions. When such solutions are used, the outer portion of the nanoparticles oxidizes to form a layer of silver oxide, which in turn can produce the Ag+ form of silver, a form that is effective against microorganisms.

It should be understood that various changes and modifications to the preferred embodiments herein will be apparent to those skilled in the art. Such changes and modifications can be made without departing from the spirit and scope of this invention and without diminishing its attendant advantages. It is therefore intended that such changes and modifications be covered by the appended claims.

I claim:

1. A blood pressure cuff liner comprising:
an organosilane antimicrobial compound or a silver containing compound or particle; and
a substrate.

2. The blood pressure cuff liner of claim 1, further comprising a fastening system.

3. The blood pressure cuff liner of claim 1, wherein the organosilane antimicrobial compound is 3-(trihydroxysilyl) propyl-dimethyloctadecyl ammonium chloride.

4. The blood pressure cuff liner of claim 3, wherein the amount of 3-(trihydroxysilyl) propyl-dimethyloctadecyl ammonium chloride ranges from about 0.01% to 5.0%.

5. The blood pressure cuff liner of claim 4, further comprising a nonionic wetting agent.

6. The blood pressure cuff liner of claim 5, wherein the nonionic wetting agent comprises ethoxylated alcohol and glycol ethers.

7. The blood pressure cuff liner of claim 2, wherein the fastening system allows the liner to be secured to a patient's arm.

8. The blood pressure cuff liner of claim 7, wherein the fastening system comprises an adhesive strip.

9. The blood pressure cuff liner of claim 2, wherein the fastening system allows the liner to be secured to the blood pressure cuff.

10. The blood pressure cuff liner of claim 9, wherein the fastening system comprises an adhesive strip.

11. The blood pressure cuff liner of claim 1, wherein the substrate comprises a material selected from the group consisting of tissue paper, polypropylene, and polyethylene.

12. A blood pressure cuff liner comprising:
3-(trihydroxysilyl) propyl-dimethyloctadecyl ammonium chloride or a silver nanoparticle;
a fastening system; and,
a substrate.

13. The blood pressure cuff liner of claim 12, wherein the fastening system comprises an adhesive strip.

14. The blood pressure cuff liner of claim 12, wherein the substrate is tissue paper.

15. The blood pressure cuff liner of claim 12, wherein the substrate is polypropylene.

16. The blood pressure cuff liner of claim 12, wherein the amount of 3-(trihydroxysilyl) propyl-dimethyloctadecyl ammonium chloride ranges from about 0.01% to 5.0%.

17. A kit comprising
a plurality of blood pressure cuff liners comprising an organosilane antimicrobial compound or a silver containing compound or particle, a fastening system, and a substrate, wherein the blood pressure cuff liners are removably connected to one another to allow dispensing of the blood pressure cuff liners.

18. The kit of claim 17 further comprising a package.

19. The kit of claim 17 wherein the organosilane compound is 3-(trihydroxysilyl) propyl-dimethyloctadecyl ammonium chloride.

20. A method of reducing the possibility of infection from use of blood pressure cuffs comprising providing a blood pressure cuff liner comprising an organosilane compound or a silver containing compound or particle.

21. The method of claim 20, further comprising securing the liner to a patient.

22. The method of claim 20 further comprising attaching the liner to the blood pressure cuff.

23. The method of claim 20, wherein the organosilane compound is 3-(trihydroxysilyl) propyl-dimethyloctadecyl ammonium chloride.

24. The method of claim 23 further comprising a wetting agent.

25. The method of claim 24 wherein the wetting agent is a non-ionic wetting agent.

26. The method of claim 25 wherein the non-ionic wetting agent comprises ethoxylated alcohol and glycol ethers.

27. The blood pressure cuff liner of claims 1, wherein the silver containing compound or particle comprises silver nanoparticles.

28. The blood pressure cuff line of claim 27, wherein the concentration of silver nanoparticles ranges from about 100 to about 3000 parts per million of silver.

29. The kit of claim 17, wherein the silver containing compound or particle comprises silver nanoparticles.

30. The method of claim 20, wherein the silver containing compound or particle comprises silver nanoparticles.
